(19) United States
(12) Patent Application Publication
(10) Pub. No.: US 2024/0257340 A1
(43) Pub. Date: Aug. 1, 2024

(54) RADIATION IMAGE PROCESSING APPARATUS, RADIATION IMAGING SYSTEM, RADIATION IMAGE PROCESSING METHOD, AND NON-TRANSITORY COMPUTER-READABLE STORAGE MEDIUM

(71) Applicant: CANON KABUSHIKI KAISHA, Tokyo (JP)

(72) Inventors: Satoru KURAMOTO, Chiba (JP); Kazuma OBARA, Tokyo (JP); Daisuke YAMADA, Kanagawa (JP)

(21) Appl. No.: 18/416,968

(22) Filed: Jan. 19, 2024

(30) Foreign Application Priority Data
Jan. 30, 2023 (JP) 2023-012132

(51) Int. Cl. G06T 7/00; G06T 3/4038

(52) U.S. Cl. CPC G06T 7/0012; G06T 3/4038; G06T 2207/10072

(57) ABSTRACT

A radiation image processing apparatus including an image processor configured to perform image processing of a radiation image is provided. The apparatus includes an obtainer configured to obtain a first image processing parameter for a first radiation image and a second image processing parameter for a second radiation image, a determiner configured to determine whether the first image processing parameter and the second image processing parameter have permissible correlation, and a notification unit configured to make a notification if the determiner determines that the first image processing parameter and the second image processing parameter do not have the permissible correlation.

### BACKGROUND OF THE INVENTION

### Field of the Invention

The present invention relates to a radiation image processing apparatus, a radiation imaging system, a radiation image processing method, and a non-transitory computer-readable storage medium.

### Description of the Related Art

In the medical field, radiation image diagnosis in which a radiation image is obtained using radiation and diagnosis is conducted is widely performed. Japanese Patent Laid-Open No. 2005-021603 shows a radiation image processing apparatus that, when generating composite image data by compositing a plurality of partial radiation images, compares patient IDs and imaging apparatus model names added to the plurality of partial radiation images and determines based the additional information whether to permit composition. An error when selecting a partial radiation image to be composited can be detected by comparing the additional information.

### SUMMARY OF THE INVENTION

For a radiation image, image processing is performed in accordance with an imaging part, an imaging method, an imaging condition, and the like such that an image more suitable for diagnosis is obtained. When compositing radiation images or conducting a follow-up examination by comparing a radiation image captured in the past with a current radiation image, if the image processing method changes, an image after composition or an image newly obtained for a follow-up examination may be inappropriate for diagnosis.

Some embodiments of the present invention provide a technique advantageous in suppressing generation of an image inappropriate for diagnosis by image processing.

According to some embodiments, a radiation image processing apparatus including an image processor configured to perform image processing of a radiation image, comprising: an obtainer configured to obtain a first image processing parameter for a first radiation image and a second image processing parameter for a second radiation image; a determiner configured to determine whether the first image processing parameter and the second image processing parameter have permissible correlation; and a notification unit configured to make a notification if the determiner determines that the first image processing parameter and the second image processing parameter do not have the permissible correlation, is provided.

According to some other embodiments, a radiation image processing method for performing image processing of a radiation image, comprising: obtaining a first image processing parameter for a first radiation image and a second image processing parameter for a second radiation image; determining whether the first image processing parameter and the second image processing parameter have permissible correlation; and making a notification if it is determined that the first image processing parameter and the second image processing parameter do not have the permissible correlation, is provided.

Further features of the present invention will become apparent from the following description of exemplary embodiments (with reference to the attached drawings).

### BRIEF DESCRIPTION OF THE DRAWINGS

FIG. 1 is a view showing an example of the configuration of a radiation imaging system including a radiation image processing apparatus according to the embodiment;

FIG. 2 is a view showing an example of the configuration of the radiation image processing apparatus according to the embodiment;

FIG. 3 is a view showing an example of the relationship between image data and image processing parameters;

FIG. 4 is a view showing an example of the relationship between image data and image processing parameters;

FIG. 5 is a view showing an example of the relationship between image data and image processing parameters;

FIG. 6 is a view showing an example of the relationship between image data and image processing parameters;

FIG. 7 is a view showing an example of the relationship between radiation image data and image processing parameters;

FIG. 8 is a flowchart showing an example of image processing in long length imaging;

FIG. 9 is a flowchart showing an example of image processing in long length imaging;

FIG. 10 is a flowchart showing an example of comparison of image processing parameters in a follow-up examination;

FIG. 11 is a flowchart showing an example of comparison of image processing parameters in angiography; and

FIG. 12 is a flowchart showing an example of comparison of image processing parameters in a pneumoconiosis test.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made to an invention that requires all such features, and multiple such features may be combined as appropriate.

Furthermore, in the attached drawings, the same reference numerals are given to the same or similar configurations, and redundant description thereof is omitted.

Radiation according to the present disclosure can include not only α-rays, β-rays, and γ-rays that are beams generated by particles (including photons) emitted by radioactive decay but also beams having equal or more energy, for example, X-rays, particle rays, and cosmic rays.

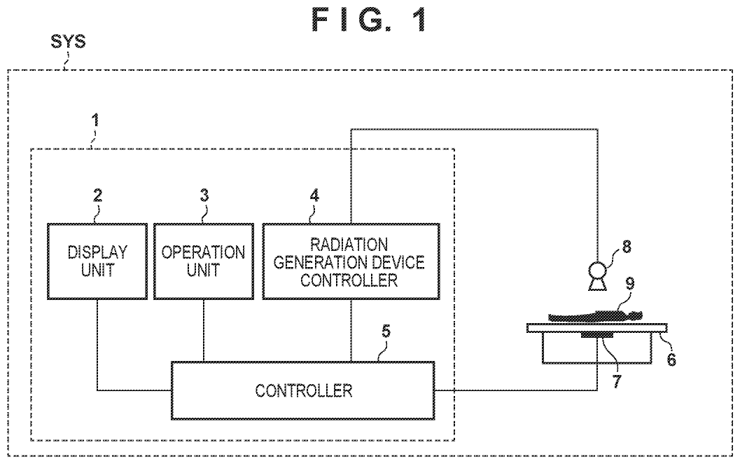

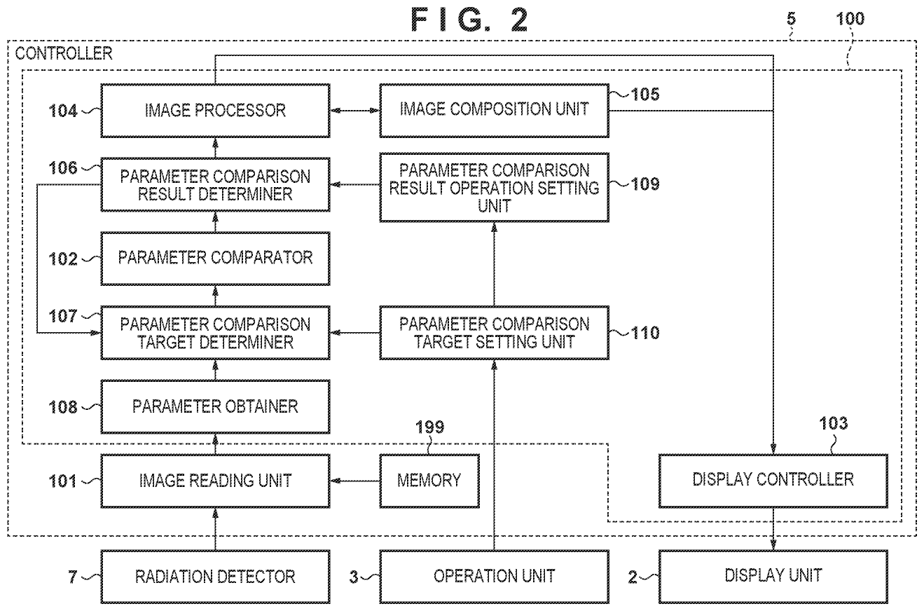

FIG. 1 is a view showing an example of the configuration of a radiation imaging system SYS including a radiation image processing apparatus according to the embodiment of the present disclosure. As shown in FIG. 1, the radiation imaging system SYS can include a radiation imaging apparatus 1 configured to perform radiation imaging, a radiation generation device 8, a radiation detector 7 that captures a radiation image by detecting radiation transmitted through an subject 9, and an imaging table 6. As will be described later with reference to FIG. 2, a radiation image processing apparatus 100 according to this embodiment is incorporated in the radiation imaging apparatus 1. However, the present invention is not limited to this, and the radiation image processing apparatus 100 may be arranged independently of the radiation imaging apparatus 1.

The radiation imaging apparatus 1 can include a display unit 2 configured to display a radiation image or various kinds of information, an operation unit 3 to be operated by an operator (for example, a radiological technologist), a radiation generation device controller 4 that controls the radiation generation device 8, and a controller 5 that controls each constituent element of the radiation imaging system. The radiation generation device controller 4 sets radiation generation conditions in the radiation generation device 8 and controls the radiation generation device 8 in radiation irradiation of the like. The radiation generation device 8 functions as a radiation source that generates radiation. The radiation generation device 8 is implemented by, for example, a radiation tube, and irradiates the subject 9 (for example, a specific part of a subject) with radiation.

The radiation detector 7 outputs image data according to radiation that has entered the radiation detector 7. Note that image data can also be referred to as a radiation image. More specifically, the radiation detector 7 detects radiation transmitted through the subject 9 as electric charges corresponding to the transmitted radiation amount. For example, a direct conversion sensor including a conversion element using a semiconductor material such as selenium and configured to directly convert radiation into electric charges may be used as the radiation detector 7. Alternatively, an indirect sensor including a scintillator using a material such as cesium iodide, and a photoelectric conversion element using a material such as silicon and configured to detect light converted from radiation by the scintillator may be used as the radiation detector 7.

The radiation detector 7 A/D-converts the detected electric charges, thereby generating image data. The generated image data may be stored in a memory (not shown) arranged in the radiation detector 7. The radiation detector 7 can add image information (an image ID, an imaging date/time, an image data transfer situation, and the like) to the image data and transfer the image information to the radiation imaging apparatus 1 together with the image data.

The image data and the image information may be stored in a memory 199 arranged in the radiation imaging apparatus 1, or may be stored in a memory (not shown) arranged in the radiation imaging system SYS provided independently of the radiation imaging apparatus 1 and the radiation detector 7. The radiation generation device controller 4, the radiation generation device 8, and the radiation detector 7 cooperatively function as a radiation imaging unit for generating image data (radiation image).

The display unit 2 is implemented by, for example, a liquid crystal display. The display unit 2 displays various kinds of information concerning the radiation imaging system SYS to the operator. The operation unit 3 is configured to include, for example, a mouse or operation buttons, transfers various kinds of instructions from the operator to the constituent elements of the radiation imaging system SYS. The operation unit 3 functions as a setting unit used by the operator to set an imaging condition of a radiation image in accordance with a procedure. In the configuration shown in FIG. 1, the display unit 2 and the operation unit 3 are illustrated as separate components. However, the present invention is not limited to this. For example, the display unit 2 and the operation unit 3 may be implemented as a touch panel that integrates these.

The controller 5 of the radiation imaging apparatus 1 is connected to the radiation detector 7 using, for example, a communication method such as a wireless LAN. The controller 5 and the radiation detector 7 transmit/receive image data and control signals to/from each other. That is, image data generated by the radiation detector 7 by radiation imaging is output (transferred) to the controller 5 using a communication method such as a wireless LAN.

The radiation imaging apparatus 1 includes the controller 5 that performs image processing for a radiation image output from the radiation detector 7 and generates a radiation image. The controller 5 has, for example, an application function operating on a computer. While controlling the operation of the radiation detector 7, the controller 5 may store the radiation image in the memory 199, output the radiation image to the display unit 2, or output a graphical user interface (GUI).

A configuration for image processing of a radiation image in the radiation imaging system SYS according to this embodiment will be described with reference to FIG. 2. First, long length composition processing including scattered ray reduction processing and noise reduction processing, which is one of image processing operations in long length imaging, will be described as an example.

As shown in FIG. 2, the controller 5 can include the radiation image processing apparatus 100 and an image reading unit 101. The image reading unit 101 reads out a radiation image received from the radiation detector 7, a radiation image stored in the memory 199 of the controller 5, an imaging condition set in accordance with a procedure, and the like, and transfers these to the radiation image processing apparatus 100 as processing targets. The image reading unit 101 also reads out, from the memory 199, image processing parameters associated with the radiation image and the imaging condition as the processing targets.

The radiation image processing apparatus 100 can include a parameter obtainer 108, a parameter comparison target determiner 107, a parameter comparison target setting unit 110, a parameter comparator 102, a parameter comparison result determiner 106, a parameter comparison result operation setting unit 109, an image processor 104, an image composition unit 105, and a display controller 103. The parameter obtainer 108 functions as an obtainer configured to obtain an already captured radiation image, an imaging condition of the radiation image, and an image processing parameter for a radiation image to be captured. The parameter comparison target determiner 107 determines whether an image processing parameter is a target to be compared by the parameter comparator 102. The parameter comparison target determiner 107 functions as an extraction unit configured to extract a parameter of a comparison target out of image processing parameters. The parameter comparison target setting unit 110 sets an image processing parameter as a target to be determined by the parameter comparison target determiner 107. The image processing parameter as the determination target may be set, for example, by the operator via the operation unit 3 or automatically in accordance with the imaging condition of a radiation image. Also, for example, the radiation image processing apparatus 100 may not include the parameter comparison target determiner 107 and the parameter comparison target setting unit 110 and set all image processing parameters for an obtained radiation image or an imaging condition as the comparison targets. The parameter comparator 102 compares the values of individual parameters as the comparison targets of image processing parameters between a plurality of radiation images or between a radiation image and the imaging condition of radiation image capturing to be performed next. The parameter comparator 102 functions as a determiner configured to determine whether image processing parameters have permissible correlation between a plurality of radiation images or between an already obtained radiation image and the imaging condition of radiation image capturing to be performed next. The parameter comparison result determiner 106 determines processing to be performed based on the result of image processing parameter comparison by the parameter comparator 102. Processing to be performed by the parameter comparison result determiner 106 is set by the parameter comparison result operation setting unit 109. The image processor 104 functions as an image processor configured to perform image processing of a radiation image. The display controller 103 outputs the radiation image that has undergone the image processing by the image processor 104 to the display unit 2. As will be described later in detail, the display controller 103 functions as a notification unit configured to make a notification via the display unit 2 if the parameter comparator 102 determines that image processing parameters do not have permissible correlation between a plurality of radiation images or between an already obtained radiation image and the imaging condition of radiation image capturing to be performed next. The image composition unit 105 functions as a composition unit configured to composite a plurality of radiation image to generate a composite image.

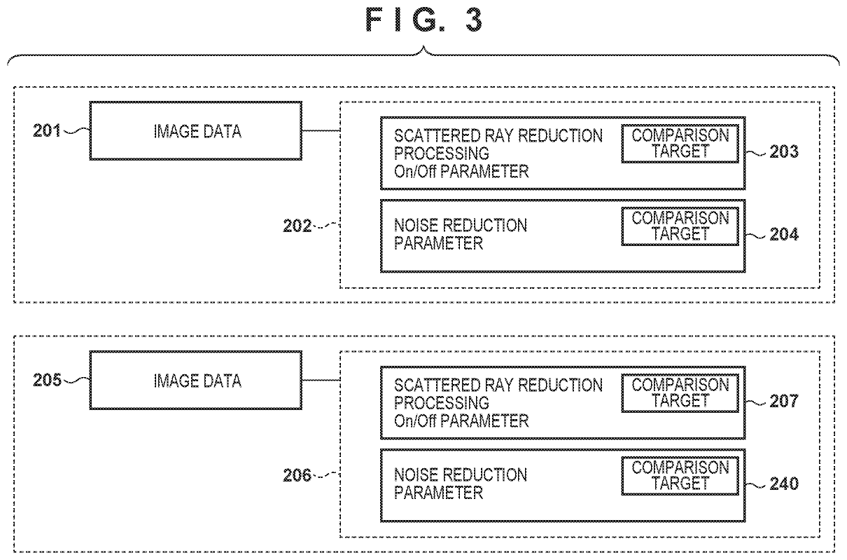

FIG. 3 is a view showing the relationship between image data (radiation images) and image processing parameters. Image data 201 and 205 are data of partial images in long length imaging, which are transferred from the radiation detector 7 to the controller 5. The image data 201 is associated with an image processing parameter 202 by the controller 5. The image data 205 is associated with an image processing parameter 206 by the controller 5.

The image processing parameter 202 and the image processing parameter 206 hold information about one or more image processing operations executable using the image processor 104. As an example, the image processing parameter 202 can include a scattered ray reduction processing On/Off parameter 203 and a noise reduction parameter 204. Similarly, the image processing parameter 206 can include a scattered ray reduction processing On/Off parameter 207 and a noise reduction parameter 240.

The scattered ray reduction processing On/Off parameters 203 and 207 are associated with the image data 201 and 205 that are partial images. The scattered ray reduction processing On/Off parameter takes a value for turning on or off scattered ray reduction processing as a setting. If scattered ray reduction processing is not executed consistently for a whole composite image (to be sometimes referred to as a long length composite image hereinafter), the possibility that the long length composite image is an inappropriate image is high. Hence, all the scattered ray reduction processing On/Off parameters associated with the image data 201 and 205 need to match. Hence, the parameter comparator 102 determines that the scattered ray reduction processing On/Off parameters have permissible correlation if all the image data 201 and 205 are only on or off. Here, as the partial images, the two image data 201 and 205 will be described as an example, but three or more image data may be used to generate a long length composite image.

As for the noise reduction parameters as well, all the parameters need to match to generate a long length composite image, like the scattered ray reduction processing On/Off parameters. For this reason, if the values of all the scattered ray reduction processing On/Off parameters associated with the image data 201 and 205 do not match, or if the values of all the noise reduction parameters do not match, the parameter comparator 102 determines that these parameters do not have permissible correlation. In this case, the parameter comparison result determiner 106 controls such that the display controller 103 notifies (to be sometimes referred to as “warns” hereinafter), via the display unit 2, that the image processing parameter 202 and the image processing parameter 206 do not have permissible correlation. Furthermore, in this case, the parameter comparison result determiner 106 may control such that the image composition unit 105 does not generate a long length composite image. That is, if the image processing parameters do not have permissible correlation, the radiation image processing apparatus 100 controls to display a warning and stop long length composition processing.

In long length imaging, the subject 9 is captured divisionally in a plurality of imaging ranges. Hence, the image data 201 and the image data 205 are captured at different timings. After that, in the radiation image processing apparatus 100, composition processing (to be sometimes referred to as long length composition processing hereinafter) is performed for a plurality of partial images (image data) received from the radiation detector 7, thereby generating a long length composite image.

A scattered ray of the scattered ray reduction processing On/Off parameters 203 and 207 indicates, out of radiation emitted from the radiation generation device 8, radiation that changes the traveling direction in the subject 9 due to interaction between the radiation and a substance when passing through the subject 9. Scattered ray reduction processing is image processing performed to estimate a scattered ray amount and improve the contrast of the image. Since a plurality of imaging processing operations are performed in long length imaging, the state of the scattered ray may change in each imaging. For this reason, scattered ray reduction processing may be performed for the partial images (image data 201 and 205) not after execution of long length composition processing but before composition.

Noise reduction processing is image processing performed to reduce random noise in a radiation image (image data). Hence, in long length imaging in which a plurality of imaging processing operations are performed, the noise amount may change because of the difference of an imaging condition in each imaging. For this reason, noise reduction processing may be performed for the partial images (image data 201 and 205) not after execution of long length composition processing but before composition.

In this embodiment, the scattered ray reduction processing On/Off parameters 203 and 207 and the noise reduction parameters 204 and 240 are set as comparison targets using the parameter comparison target setting unit 110. In addition, a setting is done using the parameter comparison result operation setting unit 109 such that if the comparison result of the image processing parameters has no permissible correlation (the image processing parameters do not match), the display unit 2 displays a warning, and the long length composition processing is stopped.

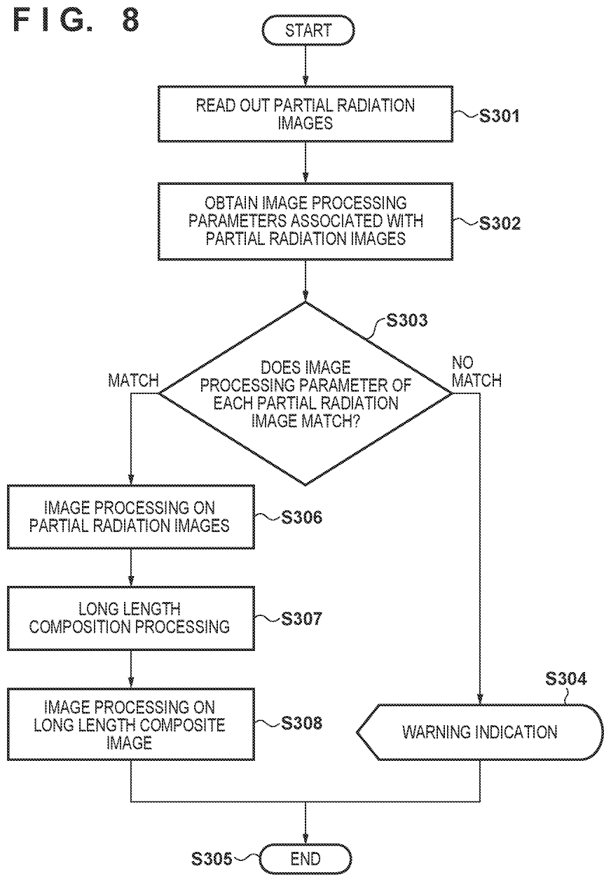

The procedure of long length imaging and long length composition processing in the radiation imaging system SYS including the radiation image processing apparatus 100 according to this embodiment will be described next with reference to the flowchart shown in FIG. 8. Under the control of the controller 5, for example, a screen for promoting the start of inspection (long length imaging) is displayed on the display unit 2. The operator starts inspection in accordance with a necessary confirmation procedure.

The operator causes the radiation generation device 8 to emit radiation as many times as the number of partial images via the radiation generation device controller 4. The radiation passes through the subject 9 and is detected by the radiation detector 7 and stored as a partial image (image data). The radiation imaging apparatus 1 receives the partial image from the radiation detector 7 using the image reading unit 101. The received partial image may be displayed on the display unit 2 via the display controller 103. Before displaying the partial image on the display unit 2, image processing such as offset processing may be performed using the image processor 104. These partial images may be stored in, for example, the memory 199.

The operator selects partial images displayed on the display unit 2, and instructs, via the operation unit 3, the radiation image processing apparatus 100 to execute long length composition processing of the partial images. When long length composition processing starts, in step S301, the target partial images (for example, the above-described image data 201 and 205) of long length imaging are read out by the image reading unit 101. This readout can be omitted if the partial images are already read out to the radiation image processing apparatus 100.

Next, in step S302, the parameter obtainer 108 obtains image processing parameters associated with the partial images. After the image processing parameters are obtained for all partial images to be composited, the process advances to step S303.

In step S303, the parameter comparator 102 compares the image processing parameters 202 and 206 associated with the partial images (image data 201 and 205). As the result of comparison, if the image processing parameters do not match, the process advances to step S304.

In step S304, using the display controller 103, the parameter comparison result determiner 106 causes the display unit 2 to display information (warning) representing that the image processing parameter 202 and the image processing parameter 206 do not have permissible correlation, that is, do not match. In this case, the parameter comparison result determiner 106 may cause, using the display controller 103, the display unit 2 to display contents for promoting an operation of correcting to make the image processing parameter 202 and the image processing parameter 206 match. Next, the process advances to step S305 to end the long length composition processing.

On the other hand, in step S303, if the image processing parameter 202 and the image processing parameter 206 match, that is, if the parameter comparator 102 determines that these have permissible correlation, the process advances to step S306. In step S306, the image processor 104 performs predetermined image processing including scattered ray reduction processing or noise reduction processing for each partial image. After the image processing for all partial images is ended, the process advances to step S307. In step S307, the image composition unit 105 performs long length composition processing using the partial images that have undergone the image processing of the image processor 104, and the process advances to step S308. That is, if the parameter comparator 102 determines that the image processing parameter 202 and the image processing parameter 206 have permissible correlation, the image processor 104 performs predetermined image processing including scattered ray reduction processing or noise reduction processing for the image data 201 and the image data 205. Next, the image composition unit 105 performs long length composition processing using the image data 201 and the image data 205, thereby generating a long length composite image.

After the long length composite image is generated, in step S308, the image processor 104 may perform image processing for the long length composite image. Then, the process advances to step S305 to end the long length composition processing.

The procedure of long length imaging and long length composition processing in which a plurality of imaging processing operations are performed to obtain partial images has been described above. The long length imaging is not limited to performing a plurality of imaging processing operations. Image processing in long length imaging performed by single radiation irradiation will be described below.

In the radiation imaging system SYS, a plurality of radiation detectors 7 are arranged under the imaging table 6, in addition to the same configuration as described above. Radiation emitted from the radiation generation device 8 is detected by the plurality of radiation detectors 7, and each of the plurality of radiation detectors 7 outputs a partial image (image data). That is, the plurality of partial images are captured at the same timing. After that, the radiation image processing apparatus 100 performs long length composition processing for the partial images transferred from the plurality of radiation detectors 7, thereby generating a long length composite image.

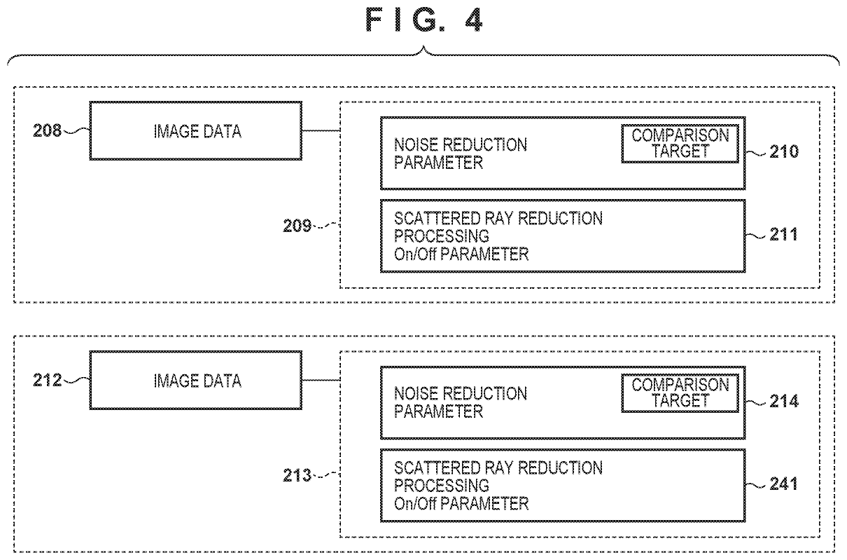

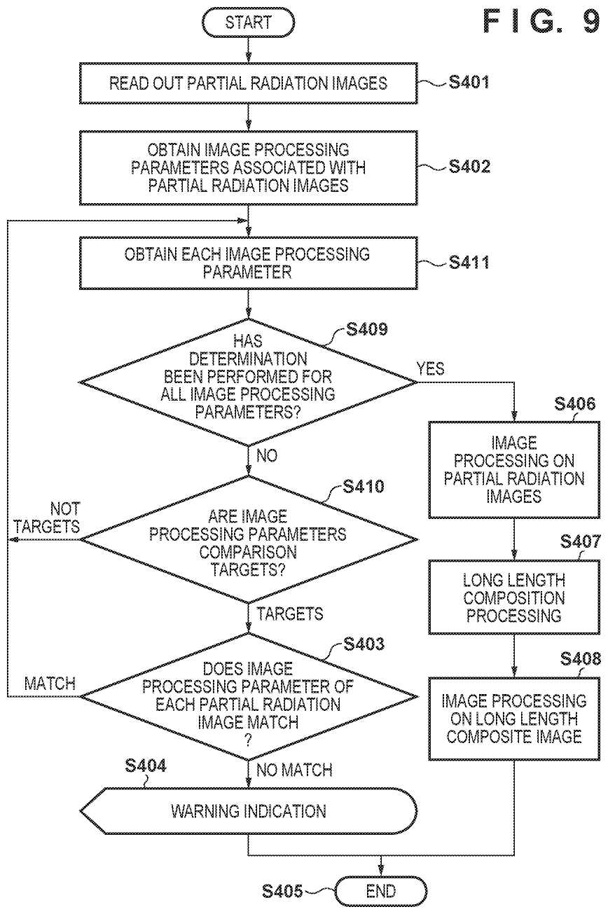

The configuration of long length imaging and long length composition processing in the radiation imaging system SYS including the radiation image processing apparatus 100 according to this embodiment will be described next with reference to FIGS. 4 and 9. Image data 208 and 212 shown in FIG. 4 are data of partial images in long length imaging, which are transferred from the radiation detector 7 to the controller 5. The image data 208 is associated with an image processing parameter 209 by the controller 5. The image data 212 is associated with an image processing parameter 213 by the controller 5.

The image processing parameter 209 and the image processing parameter 213 hold information about one or more image processing operations executable using the image processor 104. As an example, the image processing parameters 209 and 213 can include noise reduction parameters 210 and 214, respectively.

The image processor 104 receives the image data 208 and the image processing parameter 209 and performs image processing for the image data 208 in accordance with settings included in the image processing parameter 209. The noise reduction parameter 210 included in the image processing parameter 209 has not only a set value but also a value representing whether it is handled as the target of comparison processing using the parameter comparator 102. This also applies to the image data 212 and the image processing parameter 213.

Noise reduction processing is image processing for reducing random noise in a radiation image, as described above. Hence, after a long length composite image is created by composition processing, the characteristic of noise in the long length composite image may change, and the effect of noise reduction processing may lower. Hence, noise reduction processing may be performed for the partial images before composition.

On the other hand, in imaging shown in FIGS. 4 and 9, imaging is performed by one radiation irradiation. For this reason, the imaging conditions of all radiation detectors 7 match, and the states of scattered rays can match in all radiation detectors 7. Hence, unlike the above-described embodiment, scattered ray reduction processing may be performed for the long length composite image after long length composition processing.

For this reason, if all the values of the noise reduction parameters 210 and 214 associated with the partial images (image data 208 and 212) do not match, the radiation image processing apparatus 100 operates to display a warning and stop long length composition processing. On the other hand, the values of scattered ray reduction processing On/Off parameters are not the comparison targets. Hence, the parameter comparison target setting unit 110 sets the parameter comparator 102 such that the noise reduction parameter 210 is the comparison target. On the other hand, the parameter comparison target setting unit 110 sets the parameter comparator 102 such that the scattered ray reduction processing On/Off parameter is not the comparison target. In addition, a setting is done using the parameter comparison result operation setting unit 109 such that if the comparison result of the image processing parameters has no permissible correlation (the image processing parameters do not match), the display unit 2 displays a warning, and the long length composition processing is stopped.

FIG. 9 is a flowchart for explaining the procedure of long length imaging and long length composition processing in the radiation imaging system SYS including the radiation image processing apparatus 100 according to this embodiment. Under the control of the controller 5, for example, a screen for promoting the start of inspection (long length imaging) is displayed on the display unit 2. The operator starts inspection in accordance with a necessary confirmation procedure.

The operator causes the radiation generation device 8 to emit radiation via the radiation generation device controller 4. The radiation passes through the subject 9 and is detected by the plurality of radiation detectors 7 and stored as partial images (image data). The radiation imaging apparatus 1 receives the partial image from each of the plurality of radiation detectors 7 using the image reading unit 101. The received partial images may be displayed on the display unit 2 via the display controller 103. Before displaying the partial images on the display unit 2, image processing such as offset processing may be performed using the image processor 104. These partial images may be stored in, for example, the memory 199.

The radiation imaging apparatus 1 receives the images from the radiation detectors 7 using the image reading unit 101 provided in the controller 5. After that, a result of image processing performed using the image processor 104 is displayed as a partial image on the display unit 2 using the display controller 103.

The operator selects partial images displayed on the display unit 2, and instructs, via the operation unit 3, the radiation image processing apparatus 100 to execute long length composition processing of the partial images. When long length composition processing starts, in step S401, the target partial images (for example, the above-described image data 208 and 212) of long length imaging are read out by the image reading unit 101. This readout can be omitted if the partial images are already read out to the radiation image processing apparatus 100.

Next, in step S402, the parameter obtainer 108 obtains image processing parameters 209 and 213 associated with the partial images (image data 208 and 212). Next, in step S411, the noise reduction parameters 210 and 214 are obtained from the image processing parameters 209 and 213.

Step S409 is a step of judging whether comparison by the parameter comparator 102 has been executed for all the image processing parameters of comparison targets. If comparison for all the image processing parameters of comparison targets is not ended, the process advances to step S410.

In step S410, the parameter comparison target determiner 107 determines whether the obtained image processing parameters are targets to be compared using the parameter comparator 102. Since the noise reduction parameters 210 and 214 are the comparison targets, the process advances to step S403.

In step S403, the parameter comparator 102 compares the noise reduction parameters 210 and 214 included in the image processing parameters 209 and 213 associated with the partial images (image data 208 and 212). As the result of comparison, if the noise reduction parameters 210 and 214 do not match, the process advances to step S404.

In step S404, using the display controller 103, the parameter comparison result determiner 106 causes the display unit 2 to display a warning representing that the noise reduction parameter 210 and the noise reduction parameter 214 do not have permissible correlation, that is, do not match. In this case, the parameter comparison result determiner 106 may cause, using the display controller 103, the display unit 2 to display contents for promoting an operation of correcting to make the noise reduction parameter 210 and the noise reduction parameter 214 match. Next, the process advances to step S405 to end the long length composition processing.

On the other hand, if the noise reduction parameter 210 and the noise reduction parameter 214 match, that is, if the parameter comparator 102 determines that these have permissible correlation, the process returns to step S411. In step S411, scattered ray reduction processing On/Off parameters 211 and 241 are obtained from the image processing parameters 209 and 213.

Next, in step S409, since comparison for all the image processing parameters of comparison targets is not ended, the process advances to step S410.

In step S410, the parameter comparison target determiner 107 determines that the scattered ray reduction processing On/Off parameters 211 and 241 are not comparison targets, and the process returns to step S411.

In step S411, next image processing parameters are obtained, and the process advances to step S409. In step S411, if all image processing parameters are already obtained, obtaining of image processing parameters may not be performed.

The process then advances to step S409, and if comparison for all the image processing parameters of comparison targets using the parameter comparator 102 is ended, the process advances to step S406. In step S406, the image processor 104 performs predetermined image processing including noise reduction processing for the partial images (image data 208 and 212). After the image processing for all partial images is ended, the process advances to step S407. In step S407, the image composition unit 105 performs long length composition processing using the partial images that have undergone the image processing of the image processor 104, and the process advances to step S408.

In step S408, the image processor 104 performs image processing including scattered ray reduction processing for the long length composite image. If the image processing for the long length imaging is ended, the process advances to step S405 to end the long length composition processing.

In the above-described embodiment, long length imaging and long length composition processing are performed using the radiation image processing apparatus 100. For example, exemplifying imaging preparation processing including image processing parameter comparison processing in a case where two radiation images of different imaging times, which are obtained by capturing the same subject, are compared, the operation of the radiation image processing apparatus 100 according to this embodiment will be described next. This is an operation performed in a case where, for example, a follow-up examination of a body tissue of the subject 9 is conducted using the radiation imaging system SYS.

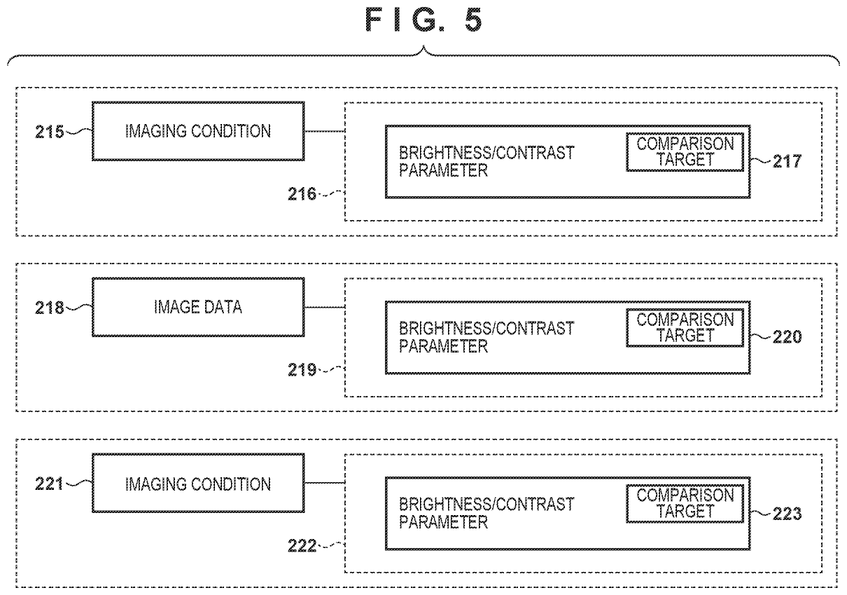

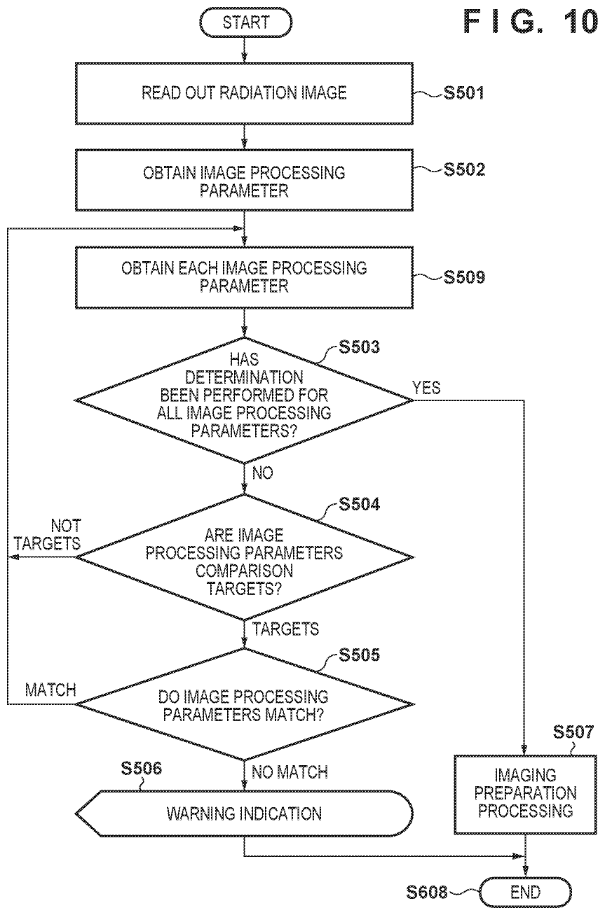

The configuration and procedure when performing imaging using the radiation imaging system SYS including the radiation image processing apparatus 100 according to this embodiment will be described with reference to FIGS. 5 and 10. Image data 218 shown in FIG. 5 is, for example, data of a radiation image already captured and transferred from the radiation detector 7 to the controller 5. For example, the image data 218 may be stored in the memory 199, or may be obtained by reading out image data stored in the memory of the radiation detector 7. In imaging conditions 215 and 221, conditions of imaging, which the operator can select in accordance with the procedure, are put together. It is assumed that after the radiation image (image data 218) is captured (for example, the next day or after a week or a month), the operator sets, via the operation unit 3, imaging conditions for imaging of radiation images to be used for a follow-up examination.

The image data 218 is associated with an image processing parameter 219 by the controller 5. The imaging conditions 215 and 221 are associated with image processing parameters 216 and 222, respectively, by the controller 5. The image processing parameters 216, 219, and 222 hold information about one or more image processing operations executable using the image processor 104. As an example, the image processing parameters 216, 219, and 222 include brightness/contrast parameters 217, 220, and 223, respectively. The brightness/contrast parameters 217, 220, and 223 include settings of image processing of brightness and contrast.

In imaging for a follow-up examination or the like, imaging preparation processing is performed. Imaging preparation processing is processing in which when an inspector performs radiation imaging, the operator sets imaging conditions for imaging of radiation images to the controller 5 via the operation unit 3, and the controller 5 prepares for radiation imaging. In imaging preparation processing, acceptance of input of imaging information input, acceptance of selection of imaging conditions, instruction of imaging condition settings to the radiation generation device controller 4, confirmation of connection to the radiation detector 7, setting of image processing parameters used in image processing to be executed for received image data (radiation image), instruction of display to the display unit 2, and the like can be performed.

In this embodiment, using the parameter comparison target setting unit 110, the brightness/contrast parameters 217, 220, and 223 are set to comparison targets. Also, a setting is done using the parameter comparison result operation setting unit 109 such that if the comparison result of the image processing parameters indicates a mismatch, a warning is displayed but the imaging preparation processing is continued.

Conventionally, the operator selects the image processing parameter 215 according to the procedure for the chest of the subject 9 as the imaging part, and performs radiation imaging and image processing using the image processing parameter 216 associated with the imaging condition 215. At this time, the image data 218 (radiation image) and the image processing parameter 219 associated with the image data 218 are obtained. A setting of image processing (image processing parameter 219) at the time of capturing of the image data 218 is added to the image data 218, and stored in, for example, the memory 199 of the controller 5.

This time, to obtain image data (radiation image) for a follow-up examination of the same subject 9, the operator sets the imaging condition (image processing parameter 222) via the operation unit 3. Also, the operator designates, via the operation unit 3, the image data 218 as image data (radiation image) to be used for comparison in the follow-up examination. According to these inputs, the controller 5 starts imaging preparation processing.

FIG. 10 is a flowchart for explaining the procedure of imaging preparation processing. When imaging preparation processing starts, first, in step S501, the past image data 218 set to the comparison target for a follow-up examination is read out.

Next, in step S502, the parameter obtainer 108 obtains the image processing parameter 219 associated with the image data 218, which is the setting of image processing when the image data 218 was captured. Also, the parameter obtainer 108 obtains the setting of image processing included in the imaging condition 221 of the radiation image selected by the operator as the image processing parameter 222 associated with the imaging condition 221.

When the image processing parameters 219 and 222 are obtained, in step S509, the brightness/contrast parameters 220 and 223 are obtained from the image processing parameters 219 and 222. After the brightness/contrast parameters 220 and 223 are obtained, the process advances to step S503.

Step S503 is a step of judging whether comparison by the parameter comparator 102 has been executed for all the image processing parameters of comparison targets. If comparison for all the image processing parameters of comparison targets is not ended, the process advances to step S504.

In step S504, the parameter comparison target determiner 107 determines whether the obtained image processing parameters are targets to be compared using the parameter comparator 102. Since the brightness/contrast parameters 220 and 223 are the comparison targets, the process advances to step S505.

In step S505, the parameter comparator 102 compares the brightness/contrast parameter 220 included in the image processing parameter 219 associated with the past image data 218 with the brightness/contrast parameter 223 included in the image processing parameter 222 associated with the imaging condition 221. As a result, if it is determined that the brightness/contrast parameter 220 and the brightness/contrast parameter 223 do not have permissible correlation (do not match), the process advances to step S506.

In step S506, using the display controller 103, the parameter comparison result determiner 106 causes the display unit 2 to display a warning representing that the brightness/contrast parameter 220 and the brightness/contrast parameter 223 do not have permissible correlation, that is, do not match. Next, the process returns to step S509. On the other hand, if the brightness/contrast parameter 220 and the brightness/contrast parameter 223 match, the process not advances to step S506 but returns to step S509.

In step S509, next image processing parameters are obtained, and the process advances to step S503. In step S509, if all image processing parameters are already obtained, obtaining of image processing parameters may not be performed.

In step S503, if comparison for all the image processing parameters of comparison targets using the parameter comparator 102 is ended, the process advances to step S507. If the process advances to step S507, remaining imaging preparation processing operations such as instruction of imaging condition settings to the radiation generation device controller 4 and confirmation of connection to the radiation detector 7 are executed. If each imaging preparation ends, the process advances to step S508 to end the imaging preparation processing.

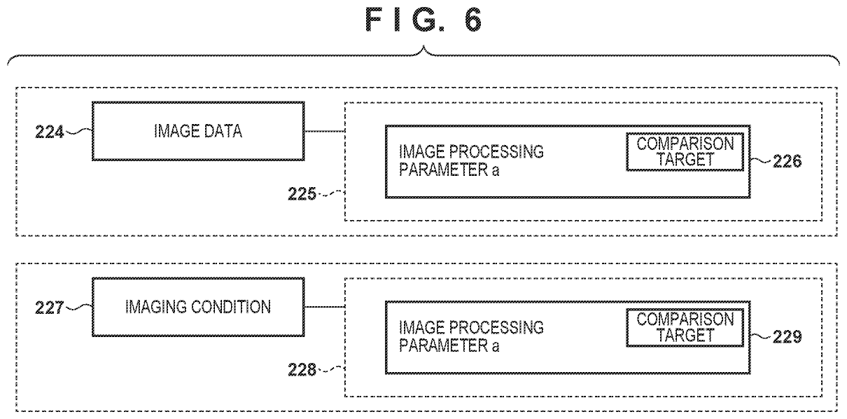

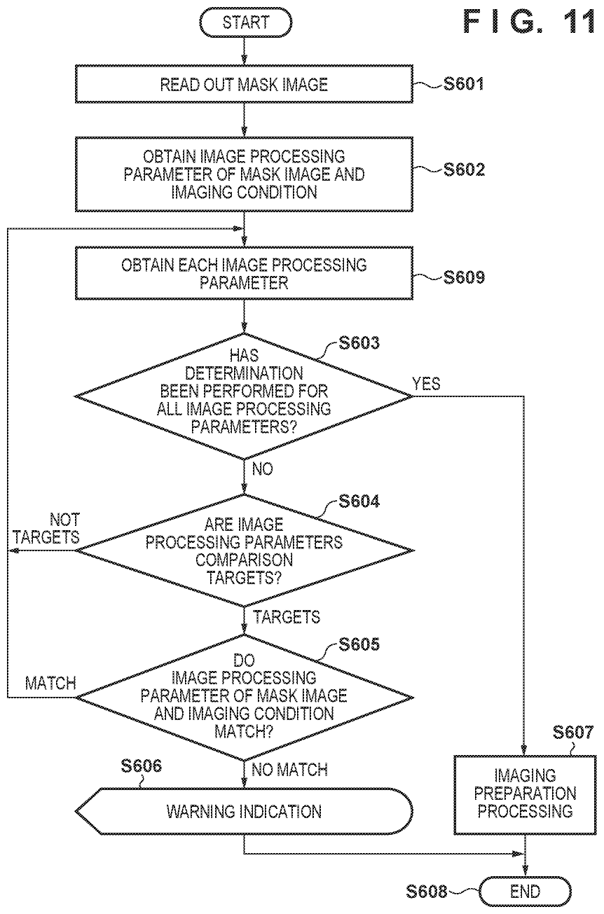

Imaging preparation processing when conducting a follow-up examination has been described with reference to FIGS. 5 and 10. Next, exemplifying imaging preparation processing including image processing parameter comparison processing in angiography using a contrast agent, the operation of the radiation image processing apparatus 100 according to this embodiment will be described with reference to FIGS. 6 and 11.

The radiation imaging system SYS including the radiation image processing apparatus 100 according to this embodiment can have the same configuration as in the above-described embodiments. Also, in this embodiment, the radiation generation device 8 can repetitively perform radiation irradiation continuously or intermittently, and the radiation detector 7 can obtain 1 to 30 radiation images (image data) in 1 sec. In addition, the image processor 104 can execute image processing of digital subtraction angiography (DSA) to be used for angiography.

Image data 224 shown in FIG. 6 is a mask image created by the controller 5 in advance. The image data 224 is associated with an image processing parameter 225. The image processing parameter 225 includes an image processing parameter a 226 necessary for image processing in an angiographic examination. An imaging condition 227 is a condition according to an imaging procedure for capturing a radiation image (live image) for which difference processing to the mask image is performed, and is associated with an image processing parameter 228. The image processing parameter 228 includes an image processing parameter a 229 necessary for image processing in an angiographic examination.

The mask image used in the angiographic examination is image data created based on a radiation image of the subject 9 before a contrast agent flows. To draw only blood vessels to which the contrast agent flows, the mask image is used to create a difference image to a radiation image (to be sometimes referred to as a live image hereinafter) captured during flowing of the contrast agent.

To observe the flowing state of the contrast agent, processing of displaying the difference image between the mask image and the continuously obtained live image needs to be performed at any time every time an image is displayed. To prevent a difference unnecessary for diagnosis from appearing in the difference image, the image processing parameters of the mask image and the live image include parameters that suitably match.

In this embodiment, the parameter comparison target setting unit 110 sets the parameter comparison target determiner 107 such that the image processing parameter a 226 included in the image processing parameter 225 associated with the mask image (image data 224) and the image processing parameter a 229 included in the image processing parameter 228 associated with the imaging condition 227 of the live image are comparison targets. In addition, a setting is done using the parameter comparison result operation setting unit 109 such that if the comparison result of the image processing parameters indicates mismatch, a warning is displayed, and the start of DSA is not permitted.

The operator arranges the subject 9 at an appropriate position, captures the subject 9, and creates the mask image from the captured image. After that, an appropriate mask image is selected via the operation unit 3, and the controller 5 is instructed to perform imaging preparation processing of the live image.

FIG. 11 is a flowchart for explaining the procedure of imaging preparation processing. First, in step S601, a mask image (image data 224) is read out.

Next, in step S602, the parameter obtainer 108 obtains the image processing parameter 225 associated with the image data 224, which is the setting of image processing when the mask image (image data 224) was captured. Also, the parameter obtainer 108 obtains the setting of image processing included in the imaging condition 227 as the image processing parameter 228 associated with the imaging condition 227.

When the image processing parameters 225 and 228 are obtained, in step S609, the image processing parameters a 226 and 229 are obtained from the image processing parameters 225 and 228. After the image processing parameters a 226 and 229 are obtained, the process advances to step S603.

Step S603 is a step of judging whether comparison by the parameter comparator 102 has been executed for all the image processing parameters of comparison targets. If comparison for all the image processing parameters of comparison targets is not ended, the process advances to step S604.

In step S604, the parameter comparison target determiner 107 determines whether the obtained image processing parameters a 226 and 229 are targets to be compared using the parameter comparator 102. Since the image processing parameters a 226 and 229 are the comparison targets, the process advances to step S605.

In step S605, the parameter comparator 102 compares the image processing parameter a 226 with the image processing parameter a 229. As a result, if it is determined that the image processing parameter a 226 and the image processing parameter a 229 do not have permissible correlation (do not match), the process advances to step S606.

In step S606, using the display controller 103, the parameter comparison result determiner 106 causes the display unit 2 to display a warning for promoting an operation of correcting to make the image processing parameter a 229 match the image processing parameter a 226. In this case, the parameter comparison result determiner 106 may cause the display unit 2 to display the image processing parameters a 226 and 229 using the display controller 103. After that, the process advances to step S608 to end the imaging preparation processing. That is, remaining imaging preparation processing operations such as instruction of imaging condition settings to the radiation generation device controller 4 and confirmation of connection to the radiation detector 7 are not executed. As a result, live image (radiation image) generation by cooperation of the radiation generation device controller 4, the radiation generation device 8, the radiation detector 7, and the like is not performed in the radiation imaging system SYS.

On the other hand, in step S605, if the image processing parameter a 226 and the image processing parameter a 229 match, the process returns to step S609. In step S609, next image processing parameters are obtained, and the process advances to step S603. In step S609, if all image processing parameters are already obtained, obtaining of image processing parameters may not be performed.

In step S603, if comparison for all the image processing parameters of comparison targets using the parameter comparator 102 is ended, the process advances to step S607. If the process advances to step S607, remaining imaging preparation processing operations such as instruction of imaging condition settings to the radiation generation device controller 4 and confirmation of connection to the radiation detector 7 are executed. If each imaging preparation ends, the process advances to step S608 to end the imaging preparation processing.

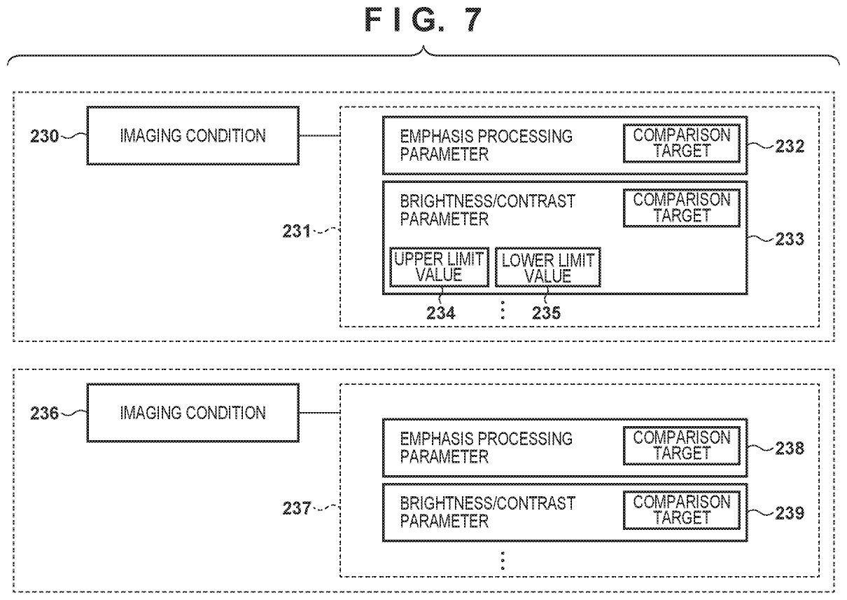

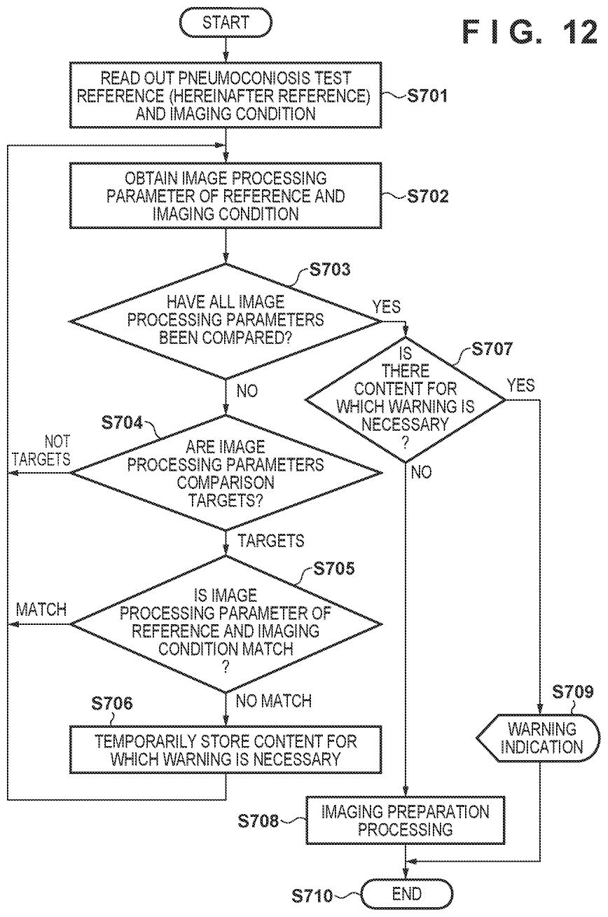

Next, exemplifying imaging preparation processing including image processing parameter comparison processing in a pneumoconiosis test, the operation of the radiation image processing apparatus 100 according to this embodiment will be described with reference to FIGS. 7 and 12. The radiation imaging system SYS including the radiation image processing apparatus 100 according to this embodiment can have the same configuration as in the above-described embodiments.

An imaging condition 230 shown in FIG. 7 is an imaging condition for setting image processing serving as the reference of a pneumoconiosis test, which is stored in the memory 199 of the controller 5, or the like, and is associated with an image processing parameter 231. In other words, the imaging condition 230 is an imaging condition for a radiation image used in a pneumoconiosis test. The setting of the imaging condition 230 may be added to a radiation image obtained by the radiation imaging system SYS for a pneumoconiosis test in the past. The image processing parameter 231 can include, for example, an emphasis processing parameter 232, a brightness/contrast parameter 233, and other image processing parameters (not shown) of image processing necessary for the pneumoconiosis test.

The emphasis processing parameter 232 is a value indicating on or off setting. Here, for example, the emphasis processing parameter 232 is set to off. The brightness/contrast parameter 233 is a numerical value and has an upper limit value 234 and a lower limit value 235. It can be determined whether the value falls within the range at the time of comparison using the parameter comparator 102. Here, for example, the upper limit value 234 is set to 17, and the lower limit value 235 is set to 14. In this embodiment, if the image processing parameters (emphasis processing parameters) match, and the value of the image processing parameter (for example, the brightness/contrast parameter) falls within a preset range, the parameter comparator 102 determines that the parameters have permissible correlation.

An imaging condition 236 is an imaging condition according to an imaging procedure used by the operator in radiation imaging for a pneumoconiosis test. The imaging condition 236 is associated with an image processing parameter 237. The image processing parameter 237 can include an emphasis processing parameter 238, a brightness/contrast parameter 239, and other image processing parameters (not shown) of image processing necessary for the pneumoconiosis test.

In this embodiment, the parameter comparison target setting unit 110 sets the parameter comparison target determiner 107 such that the emphasis processing parameters 232 and 238 and the brightness/contrast parameters 233 and 239 are comparison targets. Also, a setting is done using the parameter comparison result operation setting unit 109 such that if the comparison result of the image processing parameters indicates mismatch, a warning is displayed, and imaging preparation processing is stopped.

The operator confirms that the radiation imaging is radiation imaging necessary for a pneumoconiosis test, and selects the imaging condition 236 for the pneumoconiosis test. Next, the operator confirms whether the imaging condition 236 is a condition defined by a law. After that, the controller 5 is instructed to perform imaging preparation processing for radiation imaging.

FIG. 12 is a flowchart for explaining the procedure of imaging preparation processing. First, in step S701, the imaging condition 230 that is the pneumoconiosis test reference and the imaging condition 236 to be used in current imaging are read out. Next, the parameter obtainer 108 obtains the image processing parameter 231 associated with the imaging condition 230 and the image processing parameter 237 associated with the imaging condition 236. When the image processing parameters 231 and 237 are obtained, the process advances to step S702.

In step S702, the parameter obtainer 108 obtains the emphasis processing parameter 232 as the first image processing parameter from the image processing parameter 231 for a radiation image for the pneumoconiosis test. Also, the parameter obtainer 108 obtains the emphasis processing parameter 238 from the image processing parameter 237 as the setting of image processing included in the imaging condition 236 of a radiation image to be captured this time. After the emphasis processing parameters 232 and 238 are obtained, the process advances to step S703.

Step S703 is a step of judging whether comparison by the parameter comparator 102 has been executed for all the image processing parameters of comparison targets. If comparison for all the image processing parameters of comparison targets is not ended, the process advances to step S704.

In step S704, the parameter comparison target determiner 107 determines whether the obtained emphasis processing parameters 232 and 238 are targets to be compared using the parameter comparator 102. Since the emphasis processing parameters 232 and 238 are the comparison targets, the process advances to step S705.

In step S705, the parameter comparator 102 compares the emphasis processing parameter 232 with the emphasis processing parameter 238. As a result, if the emphasis processing parameters 232 and 238 match, the process returns to step S702. On the other hand, if it is determined that the emphasis processing parameter 232 and the emphasis processing parameter 238 do not have permissible correlation (do not match), the process advances to step S706. In step S706, the parameter comparison result determiner 106 temporarily stores, in, for example, the memory 199 of the controller 5, that the emphasis processing parameters 232 and 238 do not match (a warning is necessary), and the process returns to step S702.

If the process returns to step S702, the parameter obtainer 108 obtains the second brightness/contrast parameters 233 and 239 from the image processing parameters 231 and 237, and the process advances to step S703. In step S703, if comparison for all the image processing parameters of comparison targets is not ended, the process advances to step S704.

In step S704, the parameter comparison target determiner 107 determines whether the obtained brightness/contrast parameters 233 and 239 are targets to be compared using the parameter comparator 102. Since the obtained brightness/contrast parameters 233 and 239 are the comparison targets, the process advances to step S705.

In step S705, the parameter comparator 102 compares the brightness/contrast parameter 233 with the brightness/contrast parameter 239. As a result, if the setting of the brightness/contrast parameter 239 falls within the range between the upper limit value 234 and the lower limit value 235 of the brightness/contrast parameter 233 (has permissible correlation), the process returns to step S702.

On the other hand, if the setting of the brightness/contrast parameter 239 falls outside the range between the upper limit value 234 and the lower limit value 235 of the brightness/contrast parameter 233 (has no permissible correlation), the process advances to step S706. In step S706, that the brightness/contrast parameter 239 falls outside the range between the upper limit value 234 and the lower limit value 235 of the brightness/contrast parameter 233 (a warning is necessary) is temporarily stored in, for example, the memory 199 of the controller 5, and the process returns to step S702.

If the process returns to step S702, image processing parameters are obtained from the image processing parameters 231 and 237 again. In step S702, if all image processing parameters are already obtained, obtaining of image processing parameters may not be performed. Next, in step S703, if comparison for all the image processing parameters of comparison targets using the parameter comparator 102 is ended, the process advances to step S707.

In step S707, if the above-described steps include step S706, and that a warning is necessary is stored, the process advances to step S709. In step S709, using the display controller 103, the parameter comparison result determiner 106 causes the display unit 2 to display a warning indicating that there are image processing parameters without permissible correlation. In this case, using the display controller 103, the parameter comparison result determiner 106 may cause the display unit 2 to display the image processing parameter (for example, the emphasis processing parameter 238 or brightness/contrast parameter 239) that does not have permissible correlation (for example, does not match or falls outside the range) in the image processing parameter 237. After it is notified (warned) that the image processing parameter 237 associated with the imaging condition 236 does not have permissible correlation to the image processing parameter 231 associated with the imaging condition 230, the process advances to step S710 to end the imaging preparation processing. That is, remaining imaging preparation processing operations such as instruction of imaging condition settings to the radiation generation device controller 4 and confirmation of connection to the radiation detector 7 are not executed. As a result, the radiation generation device controller 4, the radiation generation device 8, and the radiation detector 7 do not cooperatively generate image data (radiation image) in the radiation imaging system SYS.

On the other hand, if that a warning is necessary is not stored (the above-described steps do not include step S706), the process advances to step S708. If the process advances to step S708, remaining imaging preparation processing operations such as instruction of imaging condition settings to the radiation generation device controller 4 and confirmation of connection to the radiation detector 7 are executed. If each imaging preparation ends, the process advances to step S710 to end the imaging preparation processing.

As described above, when the radiation image processing apparatus 100 according to this embodiment is used, generation of an image inappropriate for diagnosis by image processing is suppressed. As a result, it is possible to obtain the radiation imaging system SYS useful for a doctor, a radiological technologist, the subject 9 or the like.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2023-012132, filed Jan. 30, 2023, which is hereby incorporated by reference herein in its entirety.

What is claimed is:

1. A radiation image processing apparatus including an image processor configured to perform image processing of a radiation image, comprising:
an obtainer configured to obtain a first image processing parameter for a first radiation image and a second image processing parameter for a second radiation image;
a determiner configured to determine whether the first image processing parameter and the second image processing parameter have permissible correlation; and
a notification unit configured to make a notification if the determiner determines that the first image processing parameter and the second image processing parameter do not have the permissible correlation.

2. The apparatus according to claim 1, further comprising a composition unit configured to composite the first radiation image and the second radiation image to generate a composite image,
wherein if the determiner determines that the first image processing parameter and the second image processing parameter do not have the permissible correlation, the composition unit does not generate the composite image.

3. The apparatus according to claim 2, wherein if the determiner determines that the first image processing parameter and the second image processing parameter have the permissible correlation, the composition unit generates the composite image after the image processor performs image processing of the first radiation image and the second radiation image.

4. The apparatus according to claim 2, wherein the first radiation image and the second radiation image are captured at different timings, and
the first image processing parameter and the second image processing parameter each include a setting of scattered ray reduction processing and a setting of noise reduction processing.

5. The apparatus according to claim 2, wherein the first radiation image and the second radiation image are captured at the same timing, and
the first image processing parameter and the second image processing parameter each include a setting of noise reduction processing.

6. The apparatus according to claim 1, further comprising a setting unit used by an operator to set an imaging condition of the second radiation image,
wherein the imaging condition for capturing of the second radiation image is set via the setting unit after the first radiation image is captured,
a setting of image processing when the first radiation image was captured is added to the first radiation image, and
the obtainer obtains the setting of image processing when the first radiation image was captured as the first image processing parameter, and obtains a setting of image processing included in the imaging condition of the second radiation image as the second image processing parameter.

7. The apparatus according to claim 1, further comprising a setting unit used by an operator to set an imaging condition of the second radiation image,
wherein the imaging condition for capturing of the second radiation image is set via the setting unit after the first radiation image is captured, and
the obtainer obtains a setting of image processing included in an imaging condition for the first radiation image as the first image processing parameter, and obtains a setting of image processing included in the imaging condition of the second radiation image as the second image processing parameter.

8. The apparatus according to claim 7, wherein the first image processing parameter and the second image processing parameter each include a setting of emphasis processing.

9. The apparatus according to claim 6, wherein the first image processing parameter and the second image processing parameter each include a setting of image processing of brightness and contrast.

10. The apparatus according to claim 1, further comprising an extraction unit configured to extract a parameter of a comparison target out of the first image processing parameter and the second image processing parameter.

11. The apparatus according to claim 1, wherein if the first image processing parameter and the second image processing parameter match, the determiner determines that the first image processing parameter and the second image processing parameter have the permissible correlation.

12. The apparatus according to claim 1, wherein if values of the first image processing parameter and the second image processing parameter fall within a preset range, the determiner determines that the first image processing parameter and the second image processing parameter have the permissible correlation.

13. A radiation imaging system comprising:
the radiation image processing apparatus according to claim 1; and
a radiation imaging unit configured to generate a radiation image.

14. A radiation imaging system comprising:
the radiation image processing apparatus according to claim 6; and
a radiation imaging unit configured to generate a radiation image,
wherein if the determiner determines that the first image processing parameter and the second image processing parameter do not have the permissible correlation, the radiation imaging unit does not generate the second radiation image.

15. A radiation image processing method for performing image processing of a radiation image, comprising:
obtaining a first image processing parameter for a first radiation image and a second image processing parameter for a second radiation image;
determining whether the first image processing parameter and the second image processing parameter have permissible correlation; and
making a notification if it is determined that the first image processing parameter and the second image processing parameter do not have the permissible correlation.

16. A non-transitory computer-readable storage medium storing a program for causing a computer to execute the method according to claim 15.
